(19) United States
(12) Patent Application Publication
(10) Pub. No.: US 2010/0326433 A1
(43) Pub. Date: Dec. 30, 2010

(54) Oral appliance for altitude sickness

(76) Inventor: Michael D. Williams, Davie, FL (US)

Correspondence Address: MICHAEL D. WILLIAMS, 10991 SW 42ND PL, DAVIE, FL, 33328, US

(21) Appl. No.: 12/658,153

(22) Filed: Feb. 3, 2010

Related U.S. Application Data
(60) Provisional application No. 61/213,649, filed on Jun. 30, 2009.

(51) Int. Cl. A61M 16/00

(52) U.S. Cl. 128/200.24

(57) ABSTRACT

A method and use of an oral appliance device for improving breathing and subsequent blood oxygenation for an individual in high altitude conditions thereby preventing or treating altitude sickness, improving altitude acclimatization, and enhancing general and athletic performance

### CROSS-REFERENCE TO RELATED APPLICATIONS

Continuation Application No.: 61/213,649, filing date: Jun. 30, 2009

### FEDERALLY SPONSORED RESEARCH

None

### SEQUENCE LISTING

None

### FIELD OF INVENTION

The present invention relates to the use of an oral appliance that can be used to improve breathing in high altitude conditions and, more particularly, to an oral appliance that will enhance respiration and blood oxygenation in such a way as to prevent or attenuate the symptoms of altitude sickness, improve altitude acclimatization, and improve altitude related general and athletic performance.

### BACKGROUND OF THE INVENTION

Breathing at Altitude

Breathing at high altitude is a problem caused by the reduced atmospheric pressure encountered as a person ascends to higher and higher elevations. Although the relative concentration of oxygen remains the same as compared to other atmospheric gasses, there is less atmospheric pressure pushing the oxygen into a person's lungs. With the resulting less oxygen absorbed into the blood a person can suffer from multiple physical impairments and problems. The most frequently encountered problem of breathing at altitude is altitude sickness.

Altitude sickness is the term for a group of physical problems occurring at high attitude. In this patent application several terms will be defined and abbreviations for those terms will be presented and used throughout. There are basically three types of attitude sickness. The most common and least severe type is acute mountain sickness (AMS). AMS is also known as high altitude sickness, altitude Illness, attitude disease, and mountain sickness. AMS is a medical condition affecting persons who ascend too rapidly to high altitude. Many travelers experience some symptoms of altitude sickness at elevations as low as 5000 feet. However, all travelers to altitudes of 8000 feet or more will experience a certain degree of high altitude related problems. AMS occurs in 20% of visitors to 7000-9000 feet altitude and over 40% of those who travel to 10,000 feet.

AMS is an illness that can affect mountain climbers, hikers, skiers, or the casual travelers who ascend to altitude too fast. AMS is most frequently seen when people rapidly reach an altitude typically above 8,000 feet (about 2,400 meters). Symptoms may include some or all of the following: headache (usually severe), rapid pulse (heart rate), hyperventilation (breathing faster, deeper, or both), shortness of breath during exertion, changed breathing pattern at night, awakening frequently at night and difficulty sleeping, increased urination, dizziness or light-headedness, lethargy, fatigue, malaise, loss of appetite, nausea or vomiting. Night time breathing difficulties and sever headaches particularly at night are major findings in AMS. Susceptible individuals may develop these conditions within the first 48 hours. Other more severe forms of attitude sickness are High Altitude Pulmonary Edema (HAPE) and High Altitude Cerebral Edema (HACE).

It is estimated that there are over 20 million overnight visitors to the high altitude regions of Colorado alone each year that could benefit from solving this problem. AMS is very common at high altitude. In the western U.S. and Canada as many as 30 million or more travelers could be affected each year. The incidence of AMS varies with location, depending on both absolute altitude reached and rate of ascent to attitude. The traveler must be aware that the AMS, HACE and HAPE incidence increases with the rate of ascent and increase altitude itself. Altitude sickness is a serious problem.

Most travelers can acclimatize to these conditions over time especially if the ascent is gradual over a period of days. Most travelers sleeping at altitudes above 9000 feet can benefit from preventative and intervention techniques. Many travelers to high altitude do not use the gradual acclimatization process. These altitude visitors are usually seeking to participate in sports at altitude such as the following: hiking, cycling, skiing, golfing, rafting, and mountain climbing. All these activities further complicate the acclimatization process. The body can adjust to the altitude over a period of time by producing more red blood cells, increasing the depth of breathing, increasing your heart rate, and increasing vascularity to the heart and other adjustments. Acclimatization in adults is possible up to about 16,000 feet but above this height a fine balance exists between damage (death) and adjustment. A misconception of the acclimatization process is that the body returns to its sea level condition or that the hypoxia (reduced oxygen) of high altitude can be nullified by acclimatization. This is not the case. For example, on arrival at 14,000 feet maximum physical performance is 80% that at sea level. At two weeks of acclamatization it is just slightly above 85%. At sea level the normal oxygen saturation of the blood is 98%; however at an altitude of 10,000 feet the oxygen saturation in healthy adults 90% or less.

Athletic Performance at Altitude

The athletic performance of individuals begins to become impaired at altitudes above 5000 feet. The problems for mountaineers are of course very much greater than for the recreational skier. First, the altitudes may be very much greater, approaching 20,000 feet and higher, and the physical condition of the climbers themselves is greatly weakened not only from the altitude but from the long-term exposure to extreme elements. In addition to detrimental effects which may be hazardous to health, changes in altitude are known to affect athletic performance. Although most travelers do not reach the highest altitudes even the tourist at altitudes above 6000 feet will notice the difficulty with breathing even with mild exertion.

Sleep at Altitude

Steep disturbed breathing is a major characteristic of Acute Mountain Sickness (AMS). Most people don't sleep well at altitude. Climbers commonly report vivid dreams, feelings of being suffocated and wake feeling un-refreshed.

There are important changes in the way we sleep at altitude that makes sleep quality poor. The night time breathing changes at altitude to a pattern called high altitude periodic breathing (HAPB). This becomes more frequent with increasing altitude. Arousals are more frequent at attitude. It is felt that this periodic breathing of sleep may significantly alter sleep patterns and the quality of steep at night at altitude is often severely decreased adding to the severity and complications of altitude sickness. The severe headache associated with AMS most often occurs at night further complicating the steep quality. HAPB is a characteristic of AMS and other altitude related conditions. So, control or management of this breathing pattern is crucial to minimizing the symptoms of AMS.

Altitude sickness, altitude acclimatization and athletic related altitude performance are all interrelated conditions affected by the decrease of atmospheric oxygen pressure as the altitude is increased.

Prior Solutions for Altitude Sickness

There are various treatment options for attitude sickness including the following:

Gradual ascent

Decent to lower attitude

Avoid exertion (minimal or no exercise for a few days)

Avoid tobacco and respiratory depressants (alcohol and other depressant drugs including, barbiturates, tranquilizers, sleeping pills and opiates).

Medications such as: Acetazolamide (Diamox), Steroids (Decadron), Ibuprofen, Nifedipine, Sildenafil, and other medications are being investigated.

Mechanical devices such as: Oxygen canisters, Hyperbaric Chambers, CPAP type machines and face masks with hoses are also being used to increase the oxygen saturation at altitude.

Hospitalization

### PRIOR ART of RELEVANCE

### Also see: INFORMATION DISCLOSURE STATEMENT

Gamow

(U.S. Pat. No. 5,467,764)

Nov. 21, 1995

teaches a portable hypobaric sleeping chamber for acclimatization to high altitude or athletic conditioning. A cylindrically-shaped hypobaric sleeping chamber with a length longer than its diameter having a size sufficient to accommodate no more than two reclining humans.

Gamow

(U.S. Pat. No. 5,398,678)

Mar. 21, 1995

discloses a hyperbaric chamber having an internal capacity sufficient to permit an exerciser to perform exercise movements therein using stationary equipment, in the shape of sphere, semi-sphere or a truncated sphere. This body chamber will simulate the higher atmospheric pressures of lower altitudes by increasing the pressure within the chamber above that of the ambient pressure. The chamber is used for endurance conditioning, to improve the athletic performance of people who live at attitudes above sea level.

Lane

(U.S. Pat. No. 5,101,819)

Apr. 7, 1992

teaches a method of introducing nitrogen into a flight training hypobaric chamber to simulate the lower oxygen concentrations at higher altitudes for fighter pilots. The chamber is of sufficient size to allow 2 or more individuals to fully enter.

Kroll

(U.S. Pat. No. 5,988,161)

May 13, 2003

teaches a portable re-breathing device using increasing levels of carbon dioxide to displace oxygen and used to acclimate individuals to higher altitudes. The device is a portable breathing gas control system used to adjust the gas concentrations going to the subjects mask.

Kotliar

(U.S. Pat. No. 5,850,833

Dec. 22, 1998

teaches an apparatus for generating oxygen-depleted air simulating an oxygen-impoverished mountain. The low oxygen air is delivered to a user for periodic hypoxic training or therapy from a combination of face mask and external canisters and hoses.

Kotliar

(U.S. Pat. No. 5,924,419)

Jul. 20, 1999

teaches respiratory mask based hypoxic short-term athletic training devices. The system produces a hypoxic gas mixture containing carbon dioxide in quantities most suitable for necessary breathing stimulation of high altitude from a system of a face mask connected to external devices via hoses.

Basped, et al.

(U.S. Pat. No. 522,972)

Apr. 21, 2009

describes a method for treating altitude related illness by dispensing oxygen from a vending machine.

Kroll

(U.S. Pat. No. 6,561,185)

May 13, 2003

A portable method and apparatus for allowing an individual to pre-adjust at sea level for future high altitudes is taught. The invention teaches the use of a small portable breathing gas control system to adjust the gas concentrations going to a subject's facemask.

Butler

(U.S. Pat No. 6,511,964)

Jan. 28, 2003

Teaches a method for Persons encountering high altitudes without being acclimatized can prevent or alleviate the symptoms of acute mountain sickness by administration of ribose, a swallowed sugar oral medication.

Wartman

(U.S. Pat. No. 6,871,645)

Mar. 29, 2005

Teaches a method for simulating high altitude conditions by various mixtures of gases through an aviator's oxygen mask.

Swenson

US Pat application No 20090131490

May 21, 2009

Teaches a method of using the medication acetazolamide and its derivatives as an orally administered compound treatment for pulmonary disease and altitude sickness.

### Short Comings of Prior Solutions

There are many problems with these solutions for treating altitude sickness.

Gradual ascent allows the traveler to adjust to altitude without relying on medication or mechanical devices; but the process can require days or weeks to become effective. Most travelers do not have the time for this acclimatization process.

Decent to lower altitude may eliminate the symptoms of AMS but also totally eliminates the mountain experience and the reason for going to altitude. If you do not exert your self, you do not enjoy the mountain experience.

Elimination of tobacco and respiratory depressants only partially alleviates the altitude symptoms. The cause is more complex than avoidance of certain substances. Avoidance of the substances may minimize the symptoms but for AMS prone individuals the problem will still be present.

Medications have multiple side effects in particular Diamox the most widely used medication for AMS. Some Diamox Side Effects are these: an uncomfortable tingling of the fingers, toes and face, carbonated drinks tasting flat; excessive urination and rarely, blurring of vision. The other medications can cause problems with the kidneys, liver, headaches, nasal congestion and more.

Mechanical devices are cumbersome, expensive and logistically difficult to operate. Some of these devices require The use of hazardous pressurized gas cylinder containers. Many devices require the use of claustrophobic face masks, nasal cannulas, and hoses sometimes causing panic in some persons. Oxygen canisters are only a temporary solution since the benefits last only as long as the oxygen is being used.

Hospitalization will most often treat the condition but is expensive and time consuming.

The AMS solutions to date have not adequately addressed a solution that is easy to use, convenient, quick, and economical or has no side effects. The drug solutions have sometimes serious or uncomfortable side effects. Mechanical devices have not been easy or economical to use. It is suggested that high altitude visitors can benefit from an economical, simple to use, oral appliance device to treat these high altitude breathing problems. This use of an oral appliance for altitude sickness, acclimatization and enhancing altitude athletic performance and sports training has not been described in prior art.

The Oral Appliance device (OA) is a dental device or dental mouthpiece with at least one component fitting within the mouth. An oral appliance is sometimes referred to as a bite guard or a mouth piece. Most oral appliances are completely inside the mouth fitting over some part of the teeth and jaw supporting structures. While some other oral appliances may have external components outside the mouth, they will still have at least one intraoral component. One type of oral appliance is the Mandibular Advancement Device (MAD). The mandibular advancement device is an oral appliance that advances or protrudes the lower jaw (mandible). This jaw advancement or protrusion is some forward horizontal movement of the jaw as viewed with the individual in an upright vertical position. This protrusive movement is some distance beyond the normal acquired bite location. The normal bite location is a position in which all the rear teeth are in full maximum contact. At low altitudes certain types of oral appliances and in particular mandibular advancement devices (MAD) have been shown to be effective in treating sleep breathing disorders such as snoring and sleep apnea conditions. The MAD works by increasing the size of the airway. As the jaw is advanced the tongue is also pulled forward further opening the airway at the rear of the throat thereby increasing the oxygen availability during breathing. Another type of oral appliance retains the tongue in a forward position by suction or other mechanism thereby maintaining or opening the airway. The bigger opening equals more oxygen. The jaw advancement can occur only within certain physiologic limits. Since there is less available oxygen at altitude, maintaining or opening the airway for the available oxygen alleviates AMS, improves the athletic performance, and hastens acclimatization.

At low altitude the long term use of oral appliances may cause jaw soreness. However, since the oral appliance for attitude sickness is used only for a short time usually 2 to 5 days the jaw soreness issue is not a problem.

High altitude visitors can benefit from a new oral appliance device (OA) and new use that incorporates some features found in mandibular advancement devices. However, unlike the use for low altitude mandibular advancement devices the new use for this high attitude oral appliance device is to treat the physiological and pathological problems associated with low blood oxygen saturation of high altitude. Hereafter, in this application the specific high altitude oral appliance device incorporating some MAD features as welt as others to be described herein for use in high altitude conditions is called the altitude mouthpiece (AMP). This altitude mouthpiece and its new method of use for oral appliances can be used to treat attitude illnesses, AMS symptoms and other performance problems associated, with high altitude. This will allow the altitude visitor to more quickly enjoy the mountain experience while improving altitude acclimatization.

At low altitude certain mandibular advancement devices (MAD) have also been shown to be effective in treating a breathing pattern similar to High Attitude Periodic Breathing (HAPB) called Cheyne Stokes Respiration. HAPB is part of the pathology associated with AMS. Recent tests have confirmed the beneficial use of the AMP for HAPB and AMS at high altitude. The use of this AMP can treat the night time breathing problems of acute mountain sickness. This use at altitude is a new use for all oral appliances including the mandibular advancement devices and tongue retaining devices.

Treating the night time breathing problem of altitude helps alleviate or eliminate attitude illnesses and AMS. Oxygen therapy has also been shown to be effective in treating altitude sickness. Since there is less oxygen pressure at altitude there is a reduced blood saturation of oxygen. This reduced saturation causes all the symptoms of AMS and other altitude related conditions. More oxygen equals fewer problems. Any airway increase helps with AMS, since this greater opening allows more oxygen intake and blood saturation. The AMP does that. So, if the airway can be opened more, especially at high altitude during sleeping at night, the AMS symptoms are eliminated or minimized.

Pilot studies have shown the AMP to be effective in the prevention and treatment of AMS. The appliance was used mostly for night time sleeping. The effective use of the AMP eliminated the need for Diamox and the drug's many side effects such as these: numbness, tingling, or vibrating sensations in hands, feet, and lips, paresthesia and other taste alterations (carbonated drinks including sodas and beer taste very strange), increased urination and ringing in the ears. Some other less frequent side effects include: dizziness, nausea and vomiting, drowsiness, sulfonamide allergy, and headache. So any treatment to avoid medication is desirable. For those spending only a short time at altitude they want to enjoy the mountains as soon as possible without the annoying Diamox side effects. The effective use of the AMP has been shown to successfully treat AMS. Additional studies using oxygen saturation and pulse measurements have shown a reduction in pulse and oxygen episodes during sleep and a reduction in sleep arousals while using the AMP.

As a primary embodiment this AMP also called the altitude mouthpiece can help treat the conditions of altitude illness and AMS by sleeping with the appliance at night.

However, this AMP can be used anytime of the day to enhance treatment for other conditions requiring additional oxygenation at altitude. As another embodiment and method of use the altitude mouthpiece can be used for altitude treatment as an adjunct and in combination with prior art and future art including the following: Continuous Positive Air Pressure (CPAP) devices, hyperbaric and hypobaric applications, oxygen only applications, high altitude recovery chambers, altitude face masks, altitude nasal mask applications, and combined with the use of medications including but not limited to: Diamox, Sildenafil, ibuprofen, aspirin, and steroid medications.

This altitude mouthpiece application can be used in all altitude related conditions to improve oxygenation blood saturation. Another embodiment and method of use would be the athlete training regimens or exercising at altitude to increase the available oxygen for low altitude competition or high altitude activity enhancement. While the athlete remains at high altitude the AMP provides oxygen enhancement alone or in combination other art simulating low attitude oxygen blood saturation thereby increasing performance. The acclimatization process is sped up due to the increased access to oxygen while the airway is increased while using the AMP.

The altitude mouthpiece is described with respect to specific adaptations thereof, in order to demonstrate certain new uses, not heretofore available. The device is described with respect to its ability to maintain or increase the airway opening of the individual steeping or awake at high attitude. In one embodiment this AMP invention described herein, provides a unique solution to the alleviation of AMS and its related conditions. In another embodiment this AMP can be combined with other altitude illness treatment options or regimens or medications to increase their effectiveness. In another embodiment this AMP invention can be used to improve the performance of altitude exercise and general physical activity by hastening the acclimatization process. Another embodiment of this invention is to improve lower altitude exercise performance through its ability to increase the oxygenation during training at attitude or in a hyperbaric chamber. In another embodiment this AMP can alleviate the problems of AMS by providing a specific jaw position to enhance oxygenation while using the positive end-expiratory pressure breathing procedure. The daytime or nighttime use of this invention may be helpful alone or when combined with the other attitude treatment options or regimens. The disclosed uses of this altitude mouthpiece invention are novel.

### SUMMARY

In accordance with the present invention, there is provided a method and oral appliance altitude mouthpiece device for improving breathing and subsequent blood oxygenation for an individual in high attitude conditions thereby treating altitude sickness, improving altitude acclimatization, and enhancing altitude related general and athletic performance.

### Advantages of the Oral Appliance Invention

From the description above, a number of advantages of some embodiments of my oral appliance device become evident:

(a) An oral appliance device will be able to prevent and treat altitude illnesses including acute mountain sickness.

(b) This device will improve the process of altitude acclimatization.

(c) This device and method of use can improve or accelerate athletic and general performance at altitude and upon re entry to lower elevations.

(d) The method and use of this device will avoid the side effects of medications.

(e) The method and use of this device will be simple and economical.

(f) This method will be for short term use for preventing and treating altitude sickness.

(g) The method and use of this device will improve the altitude travel experience.

(h) The method and use of this device will avoid cumbersome and expensive accessory equipment and mechanical devices.

(i) The method and use of this device may avoid serious sickness and hospitalization.

(j) The method and use of this device will be safe and comfortable.

(k) The method and use of this device can be used alone and in combination with other art and other treatments for altitude sickness including but not limited to: hyperbaric enclosures, hypobaric enclosures, oxygen and gas mixtures, face and gas masks, nasal cannulas, and medications for altitude diseases.

(l) The method and use of this device can be used in combination with other art and other treatments for altitude acclimatization including but not limited to: hyperbaric enclosures, hypobaric enclosures, oxygen and other gas mixtures, face masks, gas masks, and medications for attitude diseases.

(m) The method and use of this device can be used alone and in combination with other art for attitude sports training and attitude athletic performance including but not limited to: hyperbaric enclosures, hypobaric enclosures, oxygen and other gas mixtures, face masks, gas masks, and medications for altitude athletic performance.

(n) The method and use of this device can be used to alleviate the problems of altitude illness including AMS by providing a specific jaw position to enhance oxygenation while using the positive end expiratory pressure breathing procedure.

It is concluded that this oral appliance device and method of use for treating altitude sickness, altitude acclimatization and enhancing athletic performance at altitude will be effective, safe, economical, readily available, comfortable, easy to use and fast acting.

Several advantages of one or more aspects of the oral appliance and method of use are to treat altitude sickness in a safe, effective, easy to use, fast acting, and comfortable method. Other advantages of one or more aspects are to accelerate altitude acclimatization. Other advantages of one or more aspects are to improve general altitude performance, altitude sports performance and training. These and other advantages of one or more aspects will become more apparent form a consideration of the ensuing description and drawings.

### DRAWINGS—FIGURES

FIG. 1 shows the inside rear perspective of the oral appliance invention from tongue location perspective looking toward the lips and outside of the mouth. The individual is standing upright in all figures.

FIG. 2 shows the right side perspective of the oral appliance invention in accordance with the invention's particular forward (horizontal) positioning.

FIG. 3 shows the front perspective of the oral appliance invention as it shows the particular vertical (opened) positioning of the appliance.

FIG. 4 shows front detail with the teeth inserted into the appliance.

FIG. 5 shows the oral appliance device with the tongue protruding partially into the predetermined space between the upper and lower components.

FIG. 6 shows a sectional view of the face with resting location of the tongue and the resting airway location.

FIG. 7 shows the tongue protruded with the increased airway opening Sectional view of face with tongue protruded in the approximate location produced by the oral appliance device.

FIG. 8 shows a side view of all the teeth in contact

FIG. 9 shows a side view of the teeth with the jaw protruded and opened in the approximate location produced by the oral appliance device.

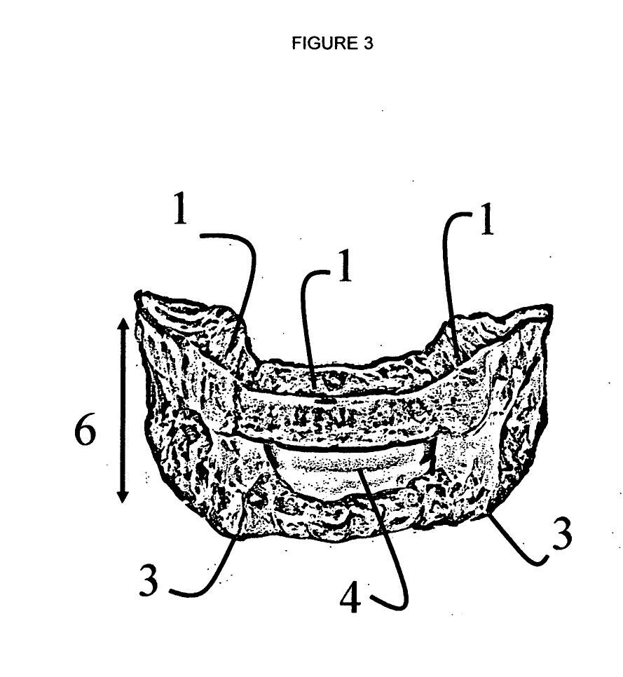

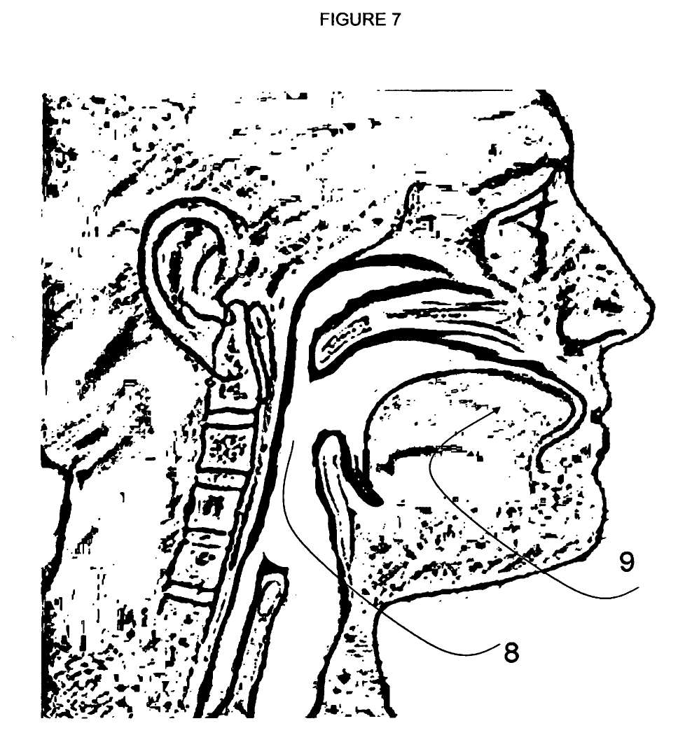

### DRAWINGS—REFERENCE NUMERALS

| | | | |
|---|---|---|---|
| 1 | groove into which upper teeth fit | 2 | groove in the oral appliance |
| 3 | groove into which lower teeth fit | 4 | space for tongue |
| 5 | protruded position jaw | 6 | vertical increase FIG. 3 |
| | | 6 | airway size at rest FIG. 7 |
| 7 | tongue at rest | 8 | airway opened |
| 9 | tongue forward | 10 | jaw forward |
| 11 | jaw open | 12 | jaw forward pulling tongue |

### DETAILED DESCRIPTION

In accordance with the present invention, there is provided an Oral Appliance device (OA) which when placed into the mouth while sleeping will prevent or ameliorate the symptoms of Acute Mountain Sickness (AMS). This device will increase the night time breathing functionality due to its particular size, configuration, composition, shape and position within the mouth. This new method of use of an oral appliance causes an improvement in night time breathing preventing or lessening altitude illness and AMS symptoms. This new method of use of an oral appliance causes an improvement in night time breathing and improving the altitude acclimatization process and improving other altitude related problems. This new method of use of an oral appliance causes an improvement in oxygenation, an improvement in general performance, and athletic performance and altitude training.

An oral appliance device and new method of use is disclosed.

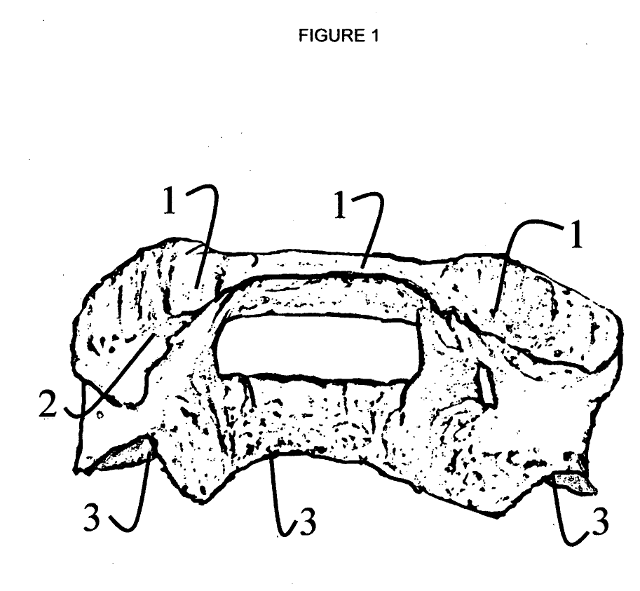

FIG. 1 shows the appearance of the oral appliance invention as viewed from tongue location perspective looking forward toward the inside of lips and then outside of the mouth. This particular high altitude oral appliance device is defined here as the Altitude Mouthpiece (AMP).The AMP oral appliance (OA) is fabricated in such a manner as to allow both the upper and lower teeth to snugly fit inside a plate, trough or groove #2. This groove is formed in such a manner that the inserted teeth and or jaw fit securely inside the appliance. This fit also allows for easy placement and removal of the appliance by the individual as required. The upper teeth fit into the top groove #1. The lower teeth fit from below into the groove #3. The groove for the lower teeth is similar in shape and configuration and fabrication to that of the upper. This certain thickness of material for the OA will allow for a comfortable tongue space. Thinner thickness may be more comfortable but may have a shorter life expectancy. Different thicknesses may be selected by the user or provider. The AMP material may be clear, opaque or colored as per the preference of the user. The overall shape of the AMP may be designed in a horse shoe shape or some other shape that wilt create a more comfortable sensation for the user. It is a preferred design that the material has a very close approximation to the teeth, gums and supporting tissue.

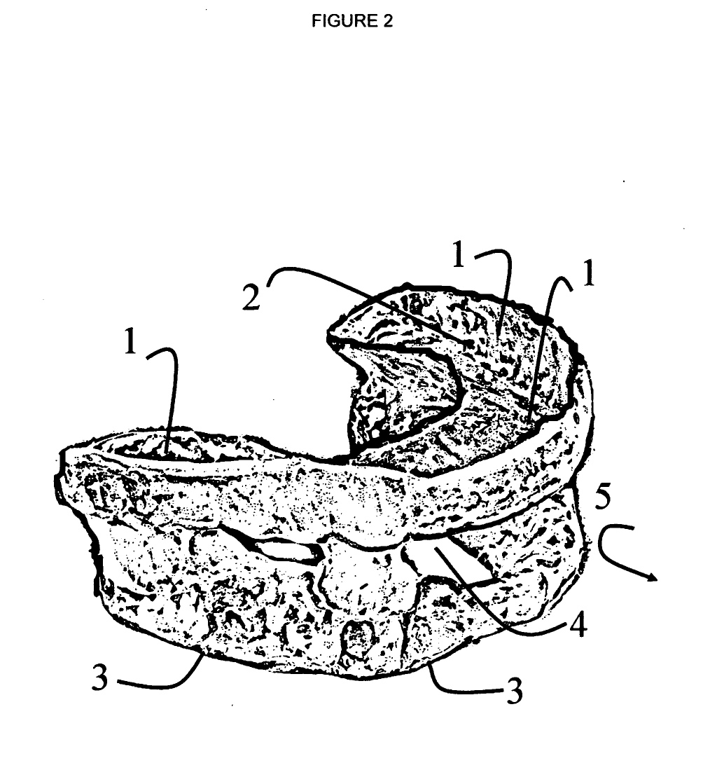

FIG. 2 shows the right side perspective of the AMP. The left side has the same appearance as this right side perspective. This figure shows the AMP invention in accordance with an approximation of the invention's particular forward horizontal positioning. The grooves #2 for upper teeth #1 and lower teeth #3 are shown here again from this perspective. This figure shows the fixed nature of the AMP. The appliance will allow for a positive insertion and seating for both the upper and lower teeth and jaws. The protrusive horizontal movement of the jaw is forward movement from a position with the individual standing upright and the head erect and head facing forward and head aligned in a correct postural position with the rest of the body. All other descriptions of oral appliance positioning are described from this orientation. Once the teeth are completely seated inside the AMP, the jaw will be property positioned.

The oral appliance device is comprised of at least one or more parts. There is provided a plate and groove for the upper teeth and jaw and a plate and groove for the lower teeth and jaw. As a single part device the plate components for the upper and lower jaw are rigidly connected by any means. A two or more part oral appliance may be temporarily fixed together by any means during the use of the AMP. The non fixed nature of the AMP can allow for OA adjustment horizontally and or vertically.

The AMP may be made as a one part appliance or composed of two or more separate parts which can be inserted individually and then set or temporarily locked into place by various means during the use of the AMP. There will be at least one part for the upper jaw and at least one part for the lower jaw. If there are two or more parts, the parts will be able to be temporarily affixed to each other during use by connection devices readily available in the dental industry. The removal and placement of the AMP will be simple and comfortable. During use of the AMP the upper and lower components are not readily movable.

In this oral appliance side application view the upper and lower components are connected together and are used as a one part device. This one part device requires at least one permanent connection on either side of the appliance. The upper and lower components are permanently connected in this illustration at four locations, two on each side. There is a space provided in the front anterior section for the tongue #4. This is further explained in the Fabrication, Function and Operation Section. This figure shows and suggests the forward horizontal location of the lower jaw or mandibular advancement. During fabrication of the AMP this mandibular advancement location #5 may be captured to optimize the OA benefit. When the AMP is modified in this protruded advancement method the mandible is advanced. This is further explained in the Fabrication, Function and Operation Section.

FIG. 3 shows the front perspective of the AMP invention as it shows the particular vertical opened positioning of the appliance. The groove for upper teeth #1 and lower teeth #3 are shown here again from this perspective. This figure shows the one part fixed nature of the AMP. The front perspective clearly shows the space provided for the tongue #4. This figure shows the cross connected components of the upper and lower portions of the AMP. The AMP can be made without the cross connection of upper component or lower component as an alternative design of the AMP. The vertical increase #6 for FIG. 3 only is illustrated in this figure. This vertical increase is one of the possible functional components of the AMP. This is further explained in the Fabrication, Function and Operation Section.

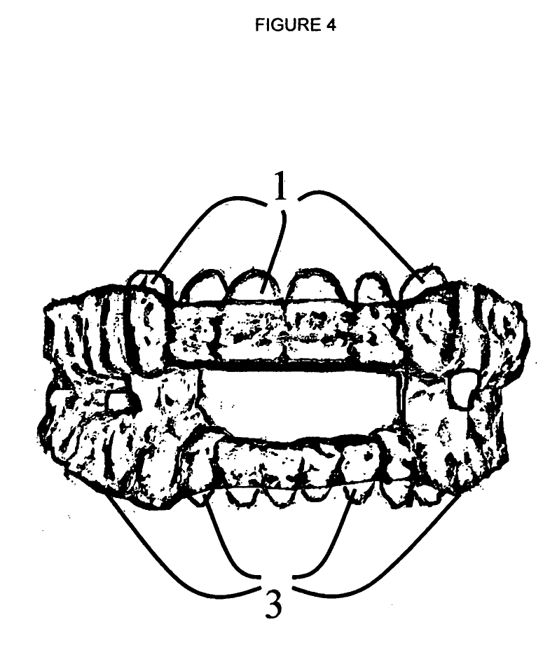

FIG. 4 shows the front detail with the oral appliance invention with the upper teeth #1 and the lower teeth #3 inserted into the appliance. This figure shows the opening of the mouth when the AMP is fully engaged into its functional position.

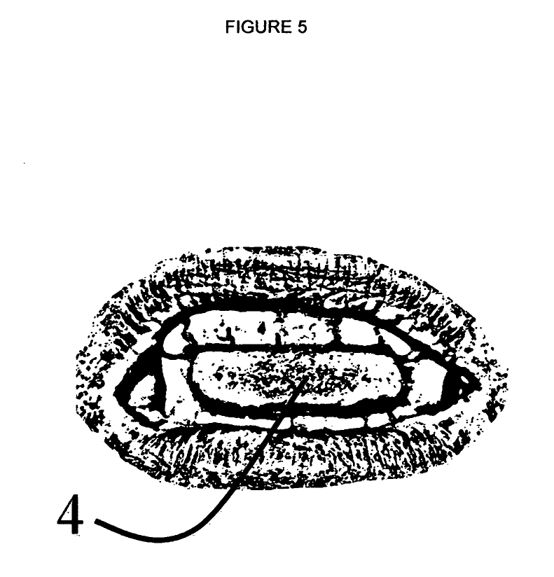

FIG. 5 shows the front perspective view of the oral appliance invention as it shows the tongue protruding into the predetermined space between the teeth. The lips do comfortably touch when the AMP is fully in place but are retracted here to show the protruded tongue #4 into the predetermined space. This is further explained in the Fabrication, Function and Operation Section.

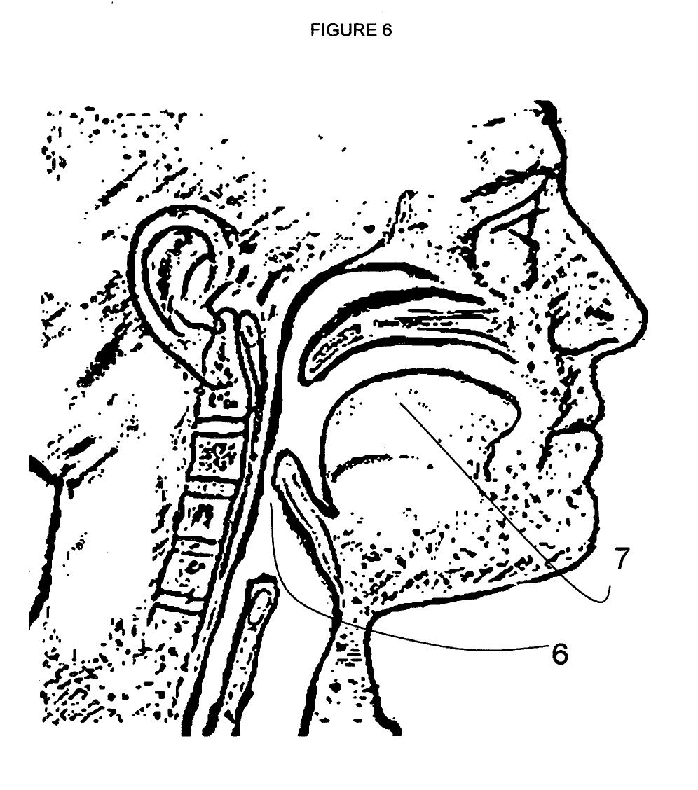

FIG. 6 shows a sectional view of the face and tongue position when the face is in its resting normal location. The airway opening #6 for FIG. 6 only at the rear of the mouth is shown and the side view of the tongue resting #7 is shown.

FIG. 7 shows the sectional side view of the face with the tongue positioned forward. The airway opening #8 in the rear of the mouth is enlarged. The tongue position #9 as it moves forward causes the airway to enlarge. FIG. 7 simulates the method and use position when the oral appliance is in place.

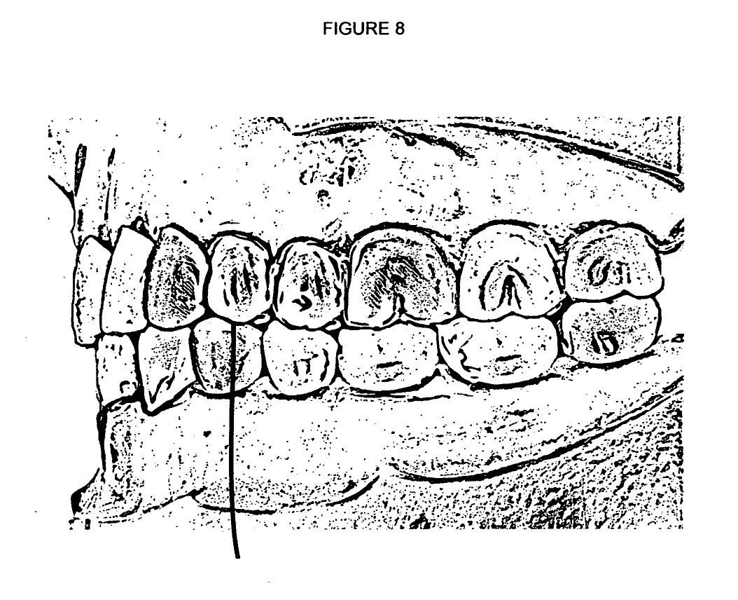

FIG. 8 shows the side view of the teeth when all the teeth are contacting #9.

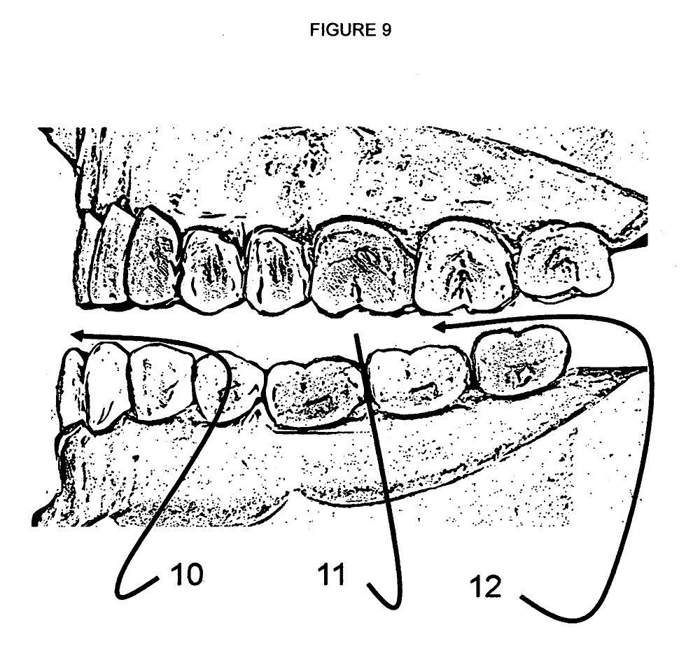

FIG. 9 shows the side view of the teeth when the jaw is opened #11 and advanced #10. This view also shows the forward advancement of the tongue #12 as it is moved forward due to its fixation to the lower jaw. FIG. 9 simulates the method and use of the oral appliance device in place.

Fabrication, Function and Operation

This oral appliance (OA) device for altitude use incorporates some features found in a generic mandibular advancement device and tongue retaining devices. This oral appliance device for use at altitude is called the altitude mouth piece (AMP). Recent tests have shown the altitude mouthpiece to be effective in the treatment of altitude breathing problems during sleep and also attenuate or eliminate the problems of AMS and other altitude disease. Specifically, the AMP increases the night time blood oxygenation thereby improving the sleep architecture which then helps with AMS symptoms and altitude disease. The OA device is comfortably inserted in to the mouth during use. The altitude mouthpiece will incorporate at least one or more of the following features which include advancement of the lower jaw, an increase in the vertical-dimension allowing for more tongue space, an anterior opening of the appliance to retain and stabilize the tongue and a forward jaw positioning for improved breathing mechanics.

The material of the AMP is fabricated from a resin, acrylic or vinyl material or a combination of these materials which are commonly available in the dental industry. Other materials not now in use may be suitable in the future. The AMP can be made in a thickness usually not less than 0.5 millimeters and not usually greater than 5 millimeters and the material may be clear, opaque or colored. Other different thickness may be used. This AMP material can be heat processed, vacuumed processed, chemically processed, light activated processed or some combination of these processes or a process yet to be defined or developed. This OA for use at high altitude is intended to treat altitude illnesses, AMS, altitude related disorders, improve altitude acclimatization and improve altitude related athletic and general performance.

For altitude illnesses, this temporary use of this appliance is intended only for the 2 to 5 day period of altitude adjustment which is usually required by most high altitude travelers. This short term usage should eliminate the jaw soreness problems seen with other oral appliances.

The Oral Appliance for treating Acute Mountain Sickness can be custom fabricated by a dental professional or individually fitted by the consumer.

The professionally fabricated AMP will require the use of accurate dental impressions and the transfer registration record of the proper lower jaw advancement and proper vertical opening of the jaw. In general the vertical position should be 3 to 9 millimeters or more of separation between the edges of the front teeth. The lips must comfortably touch when the final oral appliance device is placed. This is verified with the bite registration in place prior to construction of the oral appliance. In general the protrusive change should be a 2 to 5 or more millimeters of movement of the lower jaw advancement to a more forward location as measured from the closed position with all the rear teeth in contact. This is most often a protrusive advancement of 60 to 80% of maximum protrusion as measured from the closed position. This is a range and could be changed depending on the tempromandibular joint and muscular implications as determined by the dentist practitioner. For the dental professional the use of a George Gauge available at http://www.somnodent.com/files/files/Dentist%20Resources/NEW%20PRESENTATION.pdf can faciliate recording this protrusive and vertical measurement. The George Gauge will allow for a more accurate measurement of the amount of jaw protrusion. If the George Gauge is not used, the dentist can have the client move the jaw forward to an edge to edge position and protrude the tongue forward just beyond the teeth about 3-9 millimeters (mm) more or less depending on the practitioner's oral evaluation. This protrusion will open the jaw and create the proper amount of vertical opening that allows for the tongue protrusion. During this tongue protrusion the lower jaw will move forward about 3 -9 millimeters more or less thereby providing the proper jaw advancement. This new protrusive and vertical jaw position is captured by the means of a bite registration material readily available to the practitioner. Studies have shown that an average of 50 percent of maximal protrusion produced a 33 percent increase in the upper airway caliber, whereas maximum protrusion resulted in almost a doubling of the upper airway cross-sectional area. Other bite registration techniques currently available and those not yet developed which advance and open the vertical bite position can be used. The registration and the impressions are used in the laboratory for fabrication of the AMP.

By properly capturing and recording the correct jaw location for the AMP the tongue will automatically move forward and this forward positioning is the mechanism for opening the airway and improving breathing. The dental professional should retry the registration in the mouth to make sure that the lips can touch when the bite has been opened vertically. The lips need to touch to allow the client to steep normally and breathe through the nose in a normal fashion. Repeat the step and retake the registration, if the lips do not touch in a relaxed manner. Each clinician will use techniques that are best suited for their skill level and the client's particular situation. Other instructions for the professional regarding bite techniques may occur in the future. It is critical however that the OA incorporate at least one of the following features of mandibular advancement, tongue retention, vertical dimension increase to treat altitude illnesses including AMS and other altitude related breathing conditions. The final oral appliance device will be composed of one or more parts. If the device is composed of two or more parts, the device can be made in a fashion that could allow for protrusive and or vertical titration adjustments to optimize the breathing functionality.

This AMP can also be fitted by the individual consumer. This AMP will be at least one piece having connection for the upper and lower teeth to fit. In this fitting process the consumer purchases from a retailer a thermoplastic OA mouthpiece and places it into hot or boiling water for a period of time to soften the material. While still warm but not hot the consumer places the OA mouth piece material into the mouth at the predetermined position as described to be about 3 to 9 millimeter of vertical opening and 3 to 9 millimeters of anterior protrusion. The consumer will practice the jaw positioning prior to mouthpiece insertion according to detailed packaging instructions generally following the guidelines for the dental professional. The tongue will be slightly protruded during this process. While still warm the consumer places the mouth piece over the teeth, molds the material to fit and adapt closely to the teeth and surrounding tissue keeping it in place until the material sets. Other instructions for the consumer regarding bite techniques may occur in the future.

This consumer fitted oral appliance device process may not be as accurate as the dentist custom flitted device. However; it may serve adequately for the short term need during the period of acute mountain sickness and the altitude acclimatization process. This consumer fitted AMP available from retailers could be very cost effective and beneficial for the casual altitude traveler. Correctly adapted this fitting could produce a comparable result to a custom fitted device. In addition to the thermo plastic appliance consumer fabrication, other chemically activated or light activated materials may be used in the future to allow the consumer to self create a suitable oral appliance for treating altitude sickness.

### CONCLUSIONS RAMIFICATIONS AND SCOPE

When placed into the mouth while sleeping this device will prevent or ameliorate the symptoms of attitude illnesses including Acute Mountain Sickness (AMS). This method of use of this device will increase the night time breathing functionality due to its particular size, configuration, composition, shape and position within the mouth. The method of use of this oral appliance will open the airway and improve the oxygen saturation of the blood thereby treating acute mountain sickness, altitude acclimatization, altitude sickness and enhance athletic training and performance at all altitudes. The method and use of this oral appliance can be used at night and or daytime to improve oxygen blood saturation, reduce the heart rate, improve performance, and reduce the symptoms of altitude related illnesses.

The present AMP invention may of course be carried out in other specific ways than those herein set forth without departing from the essential characteristics of the invention. The present embodiments are, therefore, to be considered in all respects as illustrative and not restrictive and all changes coming within the meaning and equivalency range of the appended claims are intended to be embraced therein.

I claim:

1. An oral appliance device and method of use of device for the treatment of altitude illnesses including acute mountain sickness and altitude acclimatization comprising: at least one lower component fitting over the lower teeth and jaw having an attachment structure that is attached to at least one upper component fitting over the upper teeth and jaw; said components being fixed to each other while inside the mouth during use; said components being fabricated either as a single piece device permanently affixed to the other part other or having releasable components being combined at oral placement to become a temporary single fixed device during use; said components for an individual to be used while sleeping at high altitude; said components being comfortable to avoid interference while sleeping; said components being secured by frictional retention and or hydraulic suction; said single piece device or multiple part releasable component device functioning in a similar beneficial fashion; said components functioning in a capacity to produce either mandibular advancement and or vertical jaw opening and or forward tongue positioning; said components comprising the entire device.

2. The two releasable components device of claim 1 will have a means for securing the upper and lower components to each other while sleeping then acting as a single piece device.

3. The two releasable component device of claim 1 will have a means for adjusting the relative amount of mandibular advancement and jaw protrusion and a means for adjusting the relative jaw opening in a vertical dimension.

4. Said device of claim 1 maintaining, opening and or increasing the airway of the sleeping individual and improving the blood oxygen saturation of the sleeping individual and then improving the overall sleep architecture.

5. An oral appliance device and method of use of device to be used in combination with other existing and future art for preventing and treating altitude related disorders and to improve the altitude acclimatization process for an individual to be used anytime, said device comprising: at least one lower component fitting over the lower teeth and jaw having an attachment structure that is attached to at least one upper component fitting over the upper teeth and jaw; said components being fixed to each other while inside the mouth during use; said components being fabricated either as a single piece device permanently affixed to the other part other or having releasable components being combined at oral placement to become a temporary single fixed device during use; said components for an individual to be used while sleeping at high altitude; said components being comfortable to avoid interference while sleeping; said components being secured by frictional retention and hydraulic suction; said single piece device or multiple part releasable component device functioning in a similar beneficial fashion; said components functioning in a capacity to produce either mandibular advancement and or vertical jaw opening and or forward tongue positioning; said components comprising the entire device.

6. Said device of claim 5 maintaining, opening and increasing the airway of the treated individual.

7. Said device of claim 5 improving the blood oxygen saturation of the treated individual.

8. Said device of claim 5 improving the overall sleep architecture.

9. Said device of claim 5 with fixed or releasable components being compatible in size and design to accommodate face masks and hyperbaric and hypobaric devices and other gas dispensing devices.

10. An oral appliance device and method of use to be used alone or combination with other existing and future art for enhancing athletic performance at high altitude or at lower altitudes, said device comprising: at least one lower component fitting over the lower teeth and jaw having an attachment structure that is attached to at least one upper component fitting over the upper teeth and jaw; said components being fixed to each other while inside the mouth during use; said components being fabricated either as a single piece device permanently affixed to the other part other or having releasable components being combined at oral placement to become a temporary single fixed device during use; said components for an individual to be used while sleeping at high altitude; said components to be used while training or performing; said components being comfortable; said components being secured by frictional retention and or hydraulic suction; said single piece device or multiple part releasable component device functioning in a similar beneficial fashion; said components functioning in a capacity to produce either mandibular advancement and or vertical jaw opening and or forward tongue positioning; said components comprising the entire device.

11. Said device of claim 10 with releasable components having a means for adjusting the relative amount of mandibular advancement and or jaw protrusion and or a means for adjusting the relative jaw opening in a vertical dimension improving the oxygen consumption for the athletes.

12. Said device of claim 10 opening and increasing the airway of the athlete and improving the blood oxygen saturation of athlete or attitude visitor.

13. Said device of claim 10 improving the overall sleep architecture improving athletic performance and improving recovery and improving rest.

14. Said device of claim 10 resulting in improved acclimatization accelerating athletic performance and increased hematocrit and beneficial increasing oxygen consumption to muscles at all altitudes for the athlete.

15. A method for using an oral appliance device for treating altitude illnesses including acute mountain sickness, altitude acclimatization and improving altitude athletic and general performance; said method comprising the steps of: recording the proper jaw relationship, fabricating the oral appliance device; inserting the device into the mouth comfortably and maintaining the position of the device while in use.

16. Said method of claim 15 preventing or treating altitude illnesses with improved acclimatization and or improving blood oxygen saturation and general altitude breathing mechanics and or improving sleep architecture and or improving altitude athletic and general performance and or improving recovery of altitude illnesses including the use positive end expiratory breathing for use during the day or at night.

17. Said method and use of claim 15 including all known or future oral appliance devices herein not described for preventing and or treating altitude illnesses including acute mountain sickness and or altitude acclimatization and or improving altitude related general athletic performance.
